(19) United States
(12) Patent Application Publication
(10) Pub. No.: US 2025/0099089 A1
(43) Pub. Date: Mar. 27, 2025

(54) INSTRUMENTED MINIMALLY INVASIVE SURGICAL DEVICE

(71) Applicant: Mina CHOOKHACHIZADEHMOGHADAM, San Jose, CA (US)

(72) Inventor: Mina CHOOKHACHIZADEHMOGHADAM, San Jose, CA (US)

(21) Appl. No.: 18/725,594

(22) PCT Filed: Dec. 29, 2022
(86) PCT No.: PCT/IB2022/062854

§ 371 Date: Jun. 28, 2024

Related U.S. Application Data
(60) Provisional application No. 63/295,294, filed on Dec. 30, 2021.

(51) Int. Cl. A61B 17/00; A61B 90/00

(52) U.S. Cl. CPC A61B 17/00234; A61B 90/06; A61B 2017/00039; A61B 2017/00115; A61B 2017/00305; A61B 2017/00867; A61B 2017/00929; A61B 2090/065; A61B 2090/067

(57) ABSTRACT

An instrumented minimally invasive surgical device (10), comprising an elongated tube (12) defining an inner surface (18) delimiting a lumen (20) and an opposed outer surface (22), the elongated tube (12) including a deformable segment; and a strain gauge (25), the strain gauge (25) including a conductive trace (26) provided in the elongated tube (12) between the inner and outer surfaces (18 and 22) in the deformable segment, wherein a conductance of the conductive trace (26) depends on a deformation of the deformable segment.

### FIELD OF THE INVENTION

The present invention relates to the general field of surgery, and is more particularly concerned with an instrumented minimally invasive surgical device.

### BACKGROUND

Catheters and other tube-based structures are used in a variety of medical procedures, for example in the form of catheters, delivery system, endoscopes, laparoscopy handles and inflatable balloons, among others. Detecting the magnitude, direction, and/or location of applied force on the catheter is a useful feedback that can help improving the outcome of the procedure. For example, it is known that the force applied by the catheter tip during ablation is an important parameter in achieving a successful procedure. Feedback about the shape of the catheter is also useful in assisting in positioning an effective portion that will perform a treatment. However, most cardiac ablation procedures are performed using affordable manual catheters with no force and shape sensing capabilities.

Force and shape sensing has a wide range of applications in robotic surgery. However, many currently commercialized systems include complex and expensive sensing technologies. These technologies are also sometimes implemented using relatively large components, which reduce the space available in the lumen of the tube to insert the surgical instruments that will perform the actual surgery. These disadvantages also limit the quantity of information available as the number of sensors has to be limited, either due to space or cost constraints. Furthermore, force sensing technologies typically only measure a magnitude and direction of the applied force on the tip of the catheter, while more detailed information would be useful.

On the actuating side, the need for precise movements in surgery requires relatively expensive actuators to steer catheters and similar devices.

Accordingly, there exists a need for improved instrumented minimally invasive surgical device. An object of the invention is to provide such devices.

### SUMMARY OF THE INVENTION

In a broad aspect, there is provided An instrumented minimally invasive surgical device, comprising an elongated tube defining an inner surface delimiting a lumen and an opposed outer surface, the elongated tube including a deformable segment; and a strain gauge, the strain gauge including a conductive trace provided in the elongated tube between the inner and outer surfaces in the deformable segment, wherein a conductance of the conductive trace depends on a deformation of the deformable segment.

There may also be provided an instrumented minimally invasive surgical device wherein the conductive trace is a printed conductive trace including a conductive ink.

There may also be provided an instrumented minimally invasive surgical device wherein the elongated tube includes a wall, and wherein the strain gauge includes a strip embedded in the wall, the printed conductive trace being printed on the strip.

There may also be provided an instrumented minimally invasive surgical device wherein the strain gauge is entirely embedded in the elongated tube.

There may also be provided an instrumented minimally invasive surgical device wherein the inner surface is of substantially constant diameter along the whole deformable segment.

There may also be provided an instrumented minimally invasive surgical device wherein the elongated tube includes a wall extending along the whole deformable segment and wherein a strip is bonded to the wall, the printed conductive trace being printed in the strip, the strip defining part of the inner surface.

There may also be provided an instrumented minimally invasive surgical device wherein the conductive ink is covered by a protective non-conductive layer.

There may also be provided an instrumented minimally invasive surgical device further comprising a pair of conductors electrically connected to the conductive trace spaced apart from each other, the conductors extending along the elongated tube to a free end thereof.

There may also be provided an instrumented minimally invasive surgical device further comprising a controller operatively coupled to the conductors for measuring a resistance between the two conductors, wherein the resistance is indicative of a deformation of the elongated tube.

There may also be provided an instrumented minimally invasive surgical device comprising a plurality of the strain gauges provided in the deformable segment and each having a respective resistance, the controller being operatively coupled to the strain gauges for measuring the respective resistances, the respective resistances being indicative of deformations of the elongated tube.

There may also be provided an instrumented minimally invasive surgical device wherein the controller is operative for outputting a 3D shape of the deformable segment when fed with the respective resistances. For example, and non-limitingly, the shape of the deformable catheter is specified using spatial coordinates of predetermined locations on the deformable segment. In another non-limiting example, parametric methods are used to describe the shape of the deformable segment.

There may also be provided an instrumented minimally invasive surgical device wherein the controller is operative for outputting at least one of a magnitude, orientation and location of an external contact force exerted on the deformable segment when fed with the respective resistances.

There may also be provided an instrumented minimally invasive surgical device wherein the conductive traces of the strain gauges are elongated, at least two of the conductive traces from two distinct ones of the strain gauges being oriented differently relative to the deformable segment.

There may also be provided an instrumented minimally invasive surgical device wherein the elongated tube is a steerable tube provided with one or more actuating tendons extending through one or more tendon lumen provided in the elongated tube.

There may also be provided an instrumented minimally invasive surgical device wherein the conductors extend through the one or more tendon lumen.

There may also be provided an instrumented minimally invasive surgical device wherein the strain gauge has a layered structure including: an isolating substrate layer; a sensing layer including the conductive trace and overlying the substrate layer; an interface layer including a pair of conductive contacts overlying the conductive trace in electrical connection therewith opposed to the substrate layer; and an isolating protective layer overlying the interface layer.

There may also be provided an instrumented minimally invasive surgical device wherein the conductive trace is substantially rectangular.

There may also be provided an instrumented minimally invasive surgical device wherein the conductive trace is curved.

There may also be provided an instrumented minimally invasive surgical device wherein the conductive trace is in an arm of a bridge configuration.

There may also be provided an instrumented minimally invasive surgical device further comprising a temperature compensation trace distinct from the conductive trace. For example, the temperature compensation trace is made of the same material as the conductive trace. In some embodiments, the temperature compensation trace is also deformed when the elongated tube is deformed.

In another broad aspect, there is provided a controller for a steerable catheter, the steerable catheter having an elongated tube and a tendon operatively coupled to the elongated tube for steering the steerable catheter, the controller comprising a body; a shape memory alloy (SMA) motor mounted to the body, the SMA motor including a deformable element including a SMA and operable between first and second configurations achieved by changing a temperature of the deformable element, the shape memory alloy motor having a tendon mount for mounting the tendon thereto, the tendon mount moving relative to the body when the deformable element is moved between the first and second configurations; wherein, with the elongated tube fixedly mounted to the body and the tendon mounted to the tendon mount, moving the deformable element between the first and second configurations moves the tendon relative to the elongated tube to change a configuration of the catheter.

There may also be provided a controller further comprising a stress sensor in series with the tendon between the tendon and the deformable element to measure a stress in the tendon.

There may also be provided a controller further comprising a transmission between the tendon and the deformable element to change a ratio of displacement between a mobile portion of the deformable element and the tendon.

There may also be provided a controller further comprising a biasing mechanism operatively coupled to the transmission for biasing the deformable element in a predetermined direction.

There may also be provided a controller wherein the transmission includes a pulley, the controller further comprising an angular sensor for sensing rotations of the pulley relative to the body. In other embodiments, a displacement sensor is provided in series or in parallel with the tendon, intermediate member or SMA motor for sensing displacements thereof that are ultimately indicative of displacements of the tendons.

In another broad aspect, there is provided a pressure sensor, comprising: a casing defining an enclosed cavity defining an outside surface; a first strain gauge secured to the outside surface, the first strain gauge including a first conductive trace made of a conductive ink deposited on a first substrate; a second strain gauge similar to the first strain gauge and provided in the cavity so as to be shielded from external pressure variations; wherein pressure exerted on the first strain gauge produce changes in conductance of the first conductive trace.

There may also be provided a pressure sensor wherein the first and second strain gauges are part of a half-Wheatstone bridge.

In yet another broad aspect, there is provided an instrumented minimally invasive surgical device, comprising: an elongated tube defining an inner surface delimiting a lumen and an opposed outer surface, the elongated tube including a deformable segment; and a strain gauge, the strain gauge including a conductor embedded provided in the elongated tube between the inner and outer surfaces in the deformable segment, wherein a conductance of the conductor depends on a deformation of the deformable segment.

There may also be provided an instrumented minimally invasive surgical device wherein the conductor is includes carbon nanoparticles dispersed in a non-conductive matrix.

There may also be provided an instrumented minimally invasive surgical device wherein the conductor includes a TiNi shape memory alloy,

Advantageously, the proposed device can provide a relatively large quantity of information about the device at a relatively low cost while maintaining a relatively large lumen available.

The proposed SMA based actuators are mechanically simple and relatively small, while preserving a good precision.

The present PCT application claims priority to U.S. Provisional Application No. 63/295,294, filed Dec. 30, 2021, the contents of which are incorporated herein by reference.

Other objects, advantages and features of the present invention will become more apparent upon reading of the following non-restrictive description of preferred embodiments thereof, given by way of example only with reference to the accompanying drawings.

### BRIEF DESCRIPTION OF THE DRAWINGS

In the drawings:

FIG. 1, in a perspective view, illustrates an instrumented minimally invasive surgical device;

FIG. 2, in a perspective cut-away view, illustrates the device of FIG. 1;

FIG. 3, in a transversal cross-sectional view along section line A-A of FIG. 1, illustrates a strain gauge part of the device of FIG. 1;

FIG. 4, in a transversal cross-sectional view along section line B-B of FIG. 1, illustrates the device of FIG. 1;

FIG. 5, in a longitudinal cross-sectional view intersecting section line A-A of FIG. 1, illustrates the strain gauge of FIG. 3;

FIG. 6, in a longitudinal cross-sectional view, illustrates an alternative strain gauge usable in a device similar to the device of FIG. 1;

FIG. 7, in a top plan view, illustrates a plurality of strain gauges mounted to a substrate;

FIG. 8A, in a perspective view, illustrates an insert incorporating a strain gauge similar to the strain gauge of FIG. 3;

FIG. 8B, in a perspective view, illustrates an alternative insert incorporating a strain gauge similar to the strain gauge of FIG. 3;

FIG. 8C, in a perspective view, illustrates another alternative insert incorporating a strain gauge similar to the strain gauge of FIG. 3;

FIG. 8D, in a perspective view, illustrates yet another alternative insert incorporating a strain gauge similar to the strain gauge of FIG. 3;

FIG. 8E, in a perspective view, illustrates yet another alternative insert incorporating a strain gauge similar to the strain gauge of FIG. 3;

FIG. 8F, in a perspective view, illustrates yet another alternative insert incorporating a strain gauge similar to the strain gauge of FIG. 3;

FIG. 9A, in a transversal cross-sectional view midway therethrough, illustrates the insert of FIG. 8A;

FIG. 9B, in a transversal cross-sectional view midway therethrough, illustrates the insert of FIG. 8F;

FIG. 9C, in a transversal cross-sectional view midway therethrough, illustrates the insert of FIG. 8D;

FIG. 9D, in a transversal cross-sectional view midway therethrough, illustrates an insert similar to the insert of FIG. 8D;

FIG. 9E, in a transversal cross-sectional view midway therethrough, illustrates the insert of FIG. 8D;

FIG. 10A, in a top elevation view, illustrates the insert of FIG. 8A;

FIG. 10B, in a top elevation view, illustrates an insert similar to the insert of FIG. 8A, but with a strut thereof presenting a non-rectilinear outline;

FIG. 11A, in a top elevation view, illustrates a first strut configuration usable in an insert similar to the insert of FIG. 8A;

FIG. 11B, in a top elevation view, illustrates a second strut configuration usable in an insert similar to the insert of FIG. 8A;

FIG. 11C, in a top elevation view, illustrates a third strut configuration usable in an insert similar to the insert of FIG. 8A;

FIG. 12A, in a top elevation view, illustrates a fourth strut configuration usable in an insert similar to the insert of FIG. 8A;

FIG. 12B, in a top elevation view, illustrates a fifth strut configuration usable in an insert similar to the insert of FIG. 8A;

FIG. 12C, in a top elevation view, illustrates a sixth strut configuration usable in an insert similar to the insert of FIG. 8A;

FIG. 12D, in a top elevation view, illustrates a seventh strut configuration usable in an insert similar to the insert of FIG. 8A;

FIG. 13, in a schematic view, illustrates the strain gauge used in the device of FIG. 1;

FIG. 14, side cross-sectional elevation view along section line C-C of FIG. 13, illustrates the strain gauge of FIG. 13;

FIG. 15A, in a top elevation view, illustrates a conductive trace part of the strain gauge of FIG. 13;

FIG. 15B, in a top elevation view, illustrates an alternative conductive trace usable in the strain gauge of FIG. 13;

FIG. 16A, in a top elevation view, illustrates the strain gauge of FIG. 13;

FIG. 16B, in a top elevation view, illustrates an alternative strain gauge;

FIG. 16C, in a top elevation view, illustrates an other alternative strain gauge;

FIG. 16D, in a top elevation view, illustrates yet an other alternative strain gauge;

FIG. 16E, in a top elevation view, illustrates yet an other alternative strain gauge;

FIG. 16F, in a top elevation view, illustrates yet an other alternative strain gauge;

FIG. 17A, in a schematic view, illustrates a first bridge configuration incorporating the strain gauge of FIG. 13;

FIG. 17B, in a schematic view, illustrates a second bridge configuration incorporating the strain gauge of FIG. 13;

FIG. 17C, in a schematic view, illustrates a third bridge configuration incorporating the strain gauge of FIG. 13;

FIG. 18, in a perspective view, illustrates a handle usable as a controller in a device similar to the device of FIG. 1

FIG. 19A, in a perspective cut-away view, illustrates the handle of FIG. 18 with a shape memory alloy (SMA) motor thereof in a first configuration;

FIG. 19B, in a perspective cut-away view, illustrates the handle of FIG. 18 with the SMA motor in a second configuration;

FIG. 20A, in a schematic view, illustrates a first alternative deformable element usable to replace the deformable element of the SMA motor of FIGS. 19A and 19B;

FIG. 20B, in a schematic view, illustrates a second alternative deformable element usable to replace the deformable element of the SMA motor of FIGS. 19A and 19B;

FIG. 20C, in a schematic view, illustrates a third alternative deformable element usable to replace the deformable element of the SMA motor of FIGS. 19A and 19B;

FIG. 20D, in a schematic view, illustrates a fourth alternative deformable element usable to replace the deformable element of the SMA motor of FIGS. 19A and 19B;

FIG. 20E, in a schematic view, illustrates a fifth alternative deformable element usable to replace the deformable element of the SMA motor of FIGS. 19A and 19B;

FIG. 21, in a perspective view, illustrates a transmission part of the SMA motor of FIGS. 19A and 19B;

FIG. 22A, in a schematic view, illustrates a first alternative transmission usable in handles similar to the handle of FIG. 18;

FIG. 22B, in a schematic view, illustrates a second alternative transmission usable in handles similar to the handle of FIG. 18;

FIG. 22C, in a schematic view, illustrates a third alternative transmission usable in handles similar to the handle of FIG. 18;

FIG. 22D, in a schematic view, illustrates a fourth alternative transmission usable in handles similar to the handle of FIG. 18;

FIG. 23A, in a perspective cut-away view, illustrates an alternative to the handle of FIG. 18;

FIG. 23B, in a top elevation view, illustrates a pulley acting as a transmission in the handle of FIG. 23A;

FIG. 24, in a back perspective view, illustrates the pulley of FIG. 23B;

FIG. 25, in a perspective view, illustrates a stress sensor part of the SMA motor of the handle of FIG. 18;

FIG. 26, in a side elevation view, illustrates the stress sensor of FIG. 25;

FIG. 27, in perspective view, illustrates a lever component usable as a transmission in the handle of FIG. 23A;

FIG. 28, in a side elevation view, illustrates the lever component of FIG. 27;

FIG. 29, in a longitudinal cross-sectional view midway therethrough, illustrates a handle similar to the handle of FIG. 23A incorporating the pulley of FIG. 23B and an alternative biasing element;

FIG. 30, in a schematic view, illustrates a mechanism for sensing the displacement of tendons in handles similar to the handle of FIG. 18;

FIG. 31A, in a perspective view, illustrates a manner of attaching a flexible component to other components in handles similar to the handle of FIG. 18;

FIG. 31B, in a perspective view, illustrates an other manner of attaching a flexible component to other components in handles similar to the handle of FIG. 18;

FIG. 32, in a perspective view, illustrates a manner of attaching an elongated SMA wire in handles similar to the handle of FIG. 18;

FIG. 33, in a perspective view, illustrates an other manner of attaching an elongated SMA wire in handles similar to the handle of FIG. 18;

FIG. 34, in a schematic view, illustrates a mechanism for performing fine translation of the catheter of FIG. 1 relative to its handle;

FIG. 35, in a perspective view, illustrates a user interface usable to control a steerable catheter that differs from a user interface of the handle of FIG. 18;

FIG. 36, in a perspective view, illustrates an other user interface usable to control a steerable catheter that differs from a user interface of the handle of FIG. 18;

FIG. 37, in a perspective view, illustrates an alternative motor and transmission usable in handles similar to the handle of FIG. 18;

FIG. 38, in a schematic view, illustrates a computer usable in conjunction with the device of FIG. 1 and the handle of FIG. 18;

FIG. 39A, in a transversal cross-sectional view, illustrates a catheter incorporating a pressure sensor;

FIG. 39B, in a transversal cross-sectional view, illustrates the pressure sensor of FIG. 39A;

FIG. 39C, in a perspective view, illustrates the pressure sensor of FIG. 39A;

FIG. 40, in a transversal cross-sectional view, illustrates an alternative catheter incorporating a pressure sensor;

FIG. 41A, in a longitudinal cross-sectional view, illustrates an alternative pressure sensor;

FIG. 42, in a partially exploded perspective view, illustrates the pressure sensor of FIG. 41;

FIG. 43, in a transversal cross-sectional view, illustrates an alternative catheter incorporating a pressure sensor;

FIG. 44A, in a top plan view, illustrates a PVDF piezo sensor incorporating strain gauges;

FIG. 44B, in a bottom plan view, illustrates the PVDF piezo sensor of FIG. 44A;

FIG. 45, in a perspective view, illustrates an inflatable balloon incorporating strain gauges;

FIG. 46, in a flowchart, illustrates a method of using the device of FIG. 1 to measure the compressibility of a sample, such as a tissue;

FIG. 47, in a top plan view, illustrates a strain gauge incorporating a SMA alloy wire; and

FIG. 48, in a partial perspective view with parts removed, illustrates a catheter using a SMA wire as a strain gauge.

### DETAILED DESCRIPTION

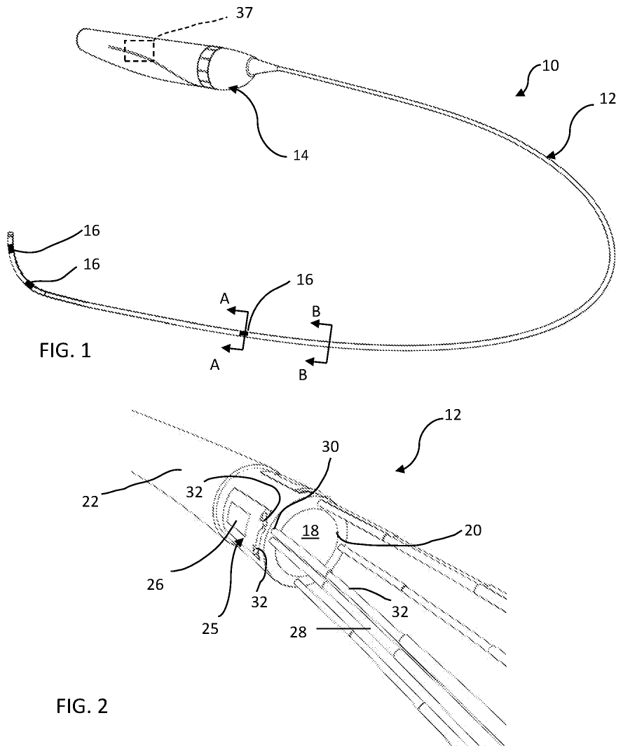

With reference to FIG. 1, there is shown an instrumented minimally invasive surgical device 10 including an elongated tube 12 mounted to a handle 14, which acts as a controller for the elongated tube 12. The elongated tube 12 illustrated in the drawings is a steerable catheter, but strain sensors described in the present document could be used in non-steerable devices, that may eventually not include the handle 14. Also, while an elongated tube 12 that is deformable along its whole length is illustrated, which is often called a catheter, the sensors and actuators described in the present document are usable in similar surgical devices that are only deformable along a portion thereof. The elongated tube 12 is provided with deformation sensors at one or more sensing locations 16. In the case in which many sensing locations 16 are provided, some are typically provided at longitudinally spaced apart locations along the elongated tube 12. Also, in some embodiments, some sensing locations 16 may be provided at circumferentially spaced apart locations around the elongated tube 12.

The elongated tube 12 includes a deformable segment. In the present embodiment, the elongated tube 12 is deformable along its entire length, and the deformable segment therefore extends along the whole length of the catheter 12. In other words, the elongated tube 12 can be bent at any location therealong. However, in alternative embodiments, only part of the elongated tube 12 is flexible enough to be deformed.

Referring to FIG. 2, the elongated tube 12 defines an inner surface 18 delimiting a lumen 20 and an opposed outer surface 22. The elongated tube 12 may be made almost entirely of a single, bulk, piece of material between the inner and outer surfaces 18 and 22, except at the sensing locations 16 as described below. However, in other embodiments, the elongated tube 12 is manufactured using different layers including different materials, or the same material, that are bond to each other or held together through friction.

Deformations of the elongated tube 12 can be passive, or, in some embodiments, active through the use of conventional tendons 28 extending through tendon lumens 30 extending along the elongated tube 12 between the inner and outer surfaces 18 and 22.

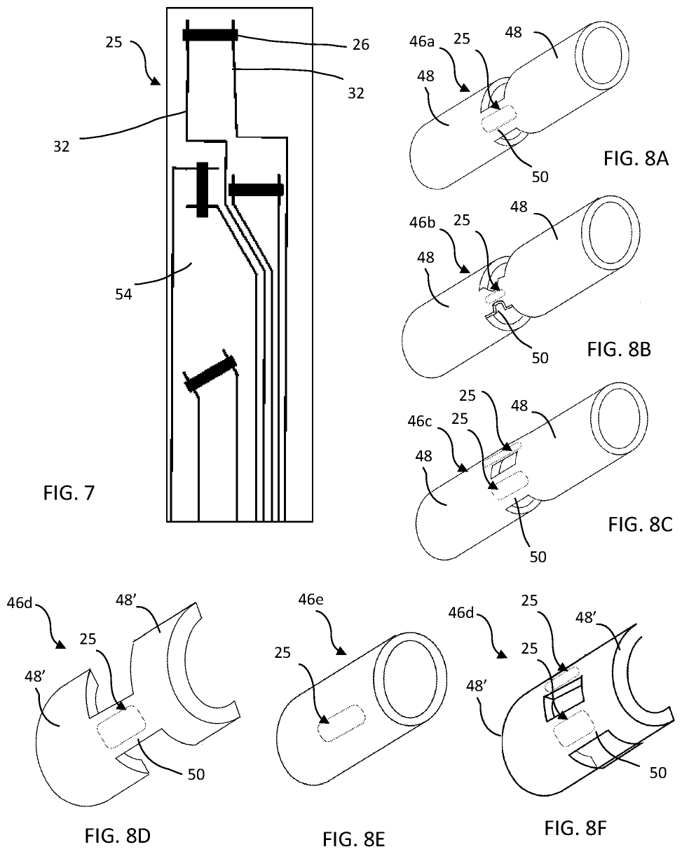

A strain gauge 25 including a conductive trace 26 is provided in the elongated tube 12 between the inner and outer surfaces 18 and 22 in the deformable segment. A conductance of the conductive trace 26 depends on a deformation of the deformable segment. Indeed, deformations of the elongated tube 12 will cause deformations of the conductive trace 26, which will induce changes in its conductance in the deformed portions thereof. Such changes in conductance can be detected by applying a voltage between two conductors 32 electrically connected to the conductive trace 26 spaced apart from each other (as seen for example in FIG. 7). the conductors 32 are typically provided in the form of wires or cables extending along the elongated tube 12 to the handle 14, and can be bare or enclosed in an insulating sheath. Some figures of the present document don't show the conductors 32 as they may be hidden or would clutter the figure too much, but it is understood that typically, such conductors are present in physical implementation of the proposed strain sensor 25. However, in some embodiments, such conductors 32 are omitted and conductance variations are detected in the strain sensor using appropriate circuits or embedded processors. This information, or the corresponding strain associated with them can then be transmitted for example through a cable or wirelessly. In the handle 14, a controller 37 is operatively coupled to the conductors 32 for measuring a resistance, or equivalently a conductance, between the two conductors 32. This resistance, which is affected by the conductance of the conductive trace 26, is indicative of a deformation of the elongated tube 12. The conductors 32 are for example metallic wires coupled to the conductive trace 26 using a conductive adhesive. In other embodiments, the conductive trace 26 is applied on a substrate on which copper or other conductors have been formed, for example using a conventional etching process, and the conductive trace 26 is applied so as to overlap the conductors 32. Other manners of coupling conductors 32 that are used to measure the conductance of conductive traces 26 are also usable.

In some embodiments, the conductive trace 26 is made of a polymer matrix doped with small amount of electrically conductive nanoparticles. In a specific non-limiting embodiment of the invention, the polymer matrix maybe made of epoxy and the nanoparticles can be made of nanocarbons formed in shape of sphere or tube (carbon nanospheres or nanotubes). Dispersion of a low quantity of nanoparticles tubes or spheres (on the order of 0.01%) results in the percolation transition, which causes the enhancement in the conductivity of the material by 10-12 orders of magnitude, when compared to the matrix alone. The film produced by depositing this material, referred here as a conductive ink, in a sensitive layer also shows a piezo resistivity behavior. In other words, its electrical conductivity changes by applying voltage or pressure.

Traditional strain gauges which are made of a metallic traces such as CuNi measure the change in resistance of the trace due to geometric effect i.e. reduction in area and increase in length of the trace or vice versa. However, in the specific embodiment including the carbon doped polymer, the quantum tunneling effect is the underlying mechanism governing the conductivity of the thin film disclosed here to measure the strain of the instrumented catheter. This change of resistance can be explained by percolation theory. In a simplified way, in this mechanism a group of dispersed particles form one or several quantum tunnel or to transmit electrons between the two conductive Ag traces. The resistance of this conductive path can be measured as indication of the applied force on the film or its deformation. This behavior can be measured to use measure strain.

The force sensing layer disclosed for the instrumental catheter in this invention offers several advantageous over traditional strain gauges. First of all, these sensors allow readily using multiple sensing elements in a significantly smaller footprint e.g. as small as 1 mm by 0.5 mm per sensing element. This is important for use in smaller diameter catheters. Secondly, they can be easily laid out in various configurations to measure the force in various directions or locations; third, they are cost effective allowing more sensors per device; fourth, they can be deposited onto various substrates such as plastics e.g. polyimide (PI) or polyester (PU), glass, metal etc; fifth, they can achieve higher resistances for a given footprint around e.g 600-2000 ohms for 1 mm by 0.5 mm footprint; and last but not the least, they can achieve a sensitivity of 8 or 10 in comparison with the sensitivity of around 2 of the traditional gauges used in this device category. Because of the flexibility of the printing process and small size of the sensor, FSL can be printed in various patterns to detect the strain at various directions as described below. The number, orientation and geometry of such printing can be designed to achieve the fit/form/function requirements for the devices at hand. Therefore, the conductive trace is not necessarily highly conductive, but instead includes a material that has a variable resistance as a function of deformation.

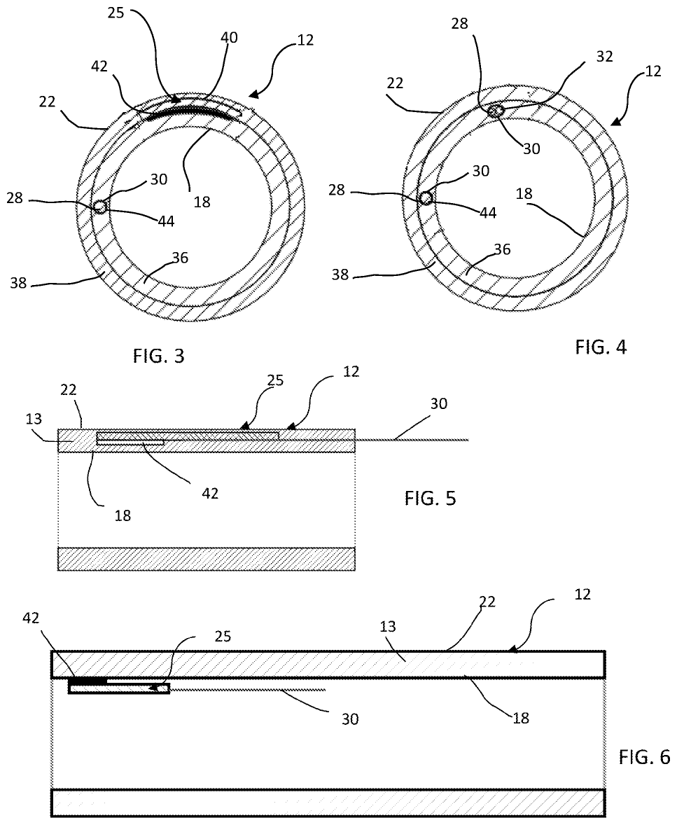

The strain gauge 25 may be integrated in the elongated tube 12 in many different manners. Referring collectively to FIGS. 3 to 5, in one embodiment, the elongated tube 12 includes a wall 13 formed by an outer layer 38 surrounding an inner layer 36 (both seen only in FIG. 3). The outer layer 38 is provided with a recess 40 opposed to the outer surface 22 receiving the strain gauge 25. The strain gauge 25 is received snugly inside the recess 40 so that the strain gauge 25 deforms with little or no hysteresis along with the elongated tube 12. In some embodiments, the strain gauge 25 is entirely or partially covered with an adhesive layer 42. The adhesive layer 42 binds the strain gauge 25 to the inner layer 36. An adhesive layer 42 may further or instead be applied between the strain gauge 25 and the outer layer 38 to bond them together. The inner and outer layers 36 and 38 can be adhered to each other or pressure fitted to each other or otherwise secured to each other using well-known methods in the art of catheter manufacturing. Also, in alternative embodiments, the adhesive layer 42 is omitted.

The tendons 28 typically slide inside a jacket 44 provided in the tendon lumens 30. As seen in FIG. 4, in some embodiments, the conductors 32 and tendons 28 may share the same tendon lumen 30. However, in alternative embodiments, the conductors are provided with their own lumen extending along the elongated tube 12. In other alternative embodiments, the tendons 28, the conductors 32, or both the tendons 28 and conductors 32 may extend through the main lumen 20 of the elongated tube 12. Other orientations are also within the scope of the invention.

In yet other embodiments, as seen in FIG. 6, the strain gauge 25 is adhered to the inside of a main wall 13 of the elongated tube 12, in a lumen of the elongated tube 12. In such embodiments, the innermost surface of the strain gauge 25 defines part of the inner surface 18. This may create a small bulge in the lumen 20. In some embodiments, this bulge could be minimized by creating a recess in the main wall 13 In yet other embodiments, the strain gauge 25 is mounted to an insert 46a to 46f seen respectively in FIGS. 8A to 8F. The inserts 46a to 46f are mountable inside the lumen 20 or wall 13 and can be made of any suitable material, such as plastic, rubber, or metals, such as aluminum, steel or shape memory alloys. The inserts 46a to 46f may be manufactured through injection molding, laser welding, 3D printing, or other manufacturing processes.

In some embodiments, as seen in FIG. 7, one or more conductive traces 26, each having a portion thereof extending between two conductors 32 may be printed on a substrate 54 using a conductive ink that is then dried. The substrate 54 takes for example the form of a relatively thin strip that may be made of a flexible material, such as a suitable non-conductive polymer. In embodiments including the recess 40, the substrate 54 and the recess 40 typically have substantially similar shapes so that the substrate 54 can be inserted in the recess 40.

The conductive traces 26 may be substantially elongated. When many strain gauges 25 are provided, either on a single substrate 54 or on many different substrates, some of the conductive traces 26 may oriented differently relative to each other, and therefore relative to the deformable segment of the elongated tube 12. For example, elongated traces 26 may extend longitudinally, circumferencially, or helically relative to the elongated tube 12.

Referring collectively to FIGS. 8A to 8F, the strain gauge 25 may be part of an insert 46a to 46f that is mountable inside the elongated tube 12 (not shown in FIGS. 8A to 8F). For example, the insert 46a, 46b, 46c, 46d and 46f includes a pair of anchors 48 or 48′ that can be mounted, for example using an adhesive, inside the elongated tube 12. The anchors 48 and 48′ are linked to each other through one or more struts 50 on which one ore more strain gauges 25 are mounted, for example using an adhesive, so as to detect deformations of these struts 50. The strain gauge 25 may also use a conductive ink directly deposited on the strut 50. The insert 46a, 46b, 46c and 46f includes for example two coaxial tubular anchors 48 of a diameter substantially similar to that of the lumen 20. The strut 50 may have a neck 52, as in the insert 46b, at the location of the strain gauge 25 to facilitated bending of the strut 50. In some embodiments, the strain gauge 25 is directly mounted or printed to a single tube 48, as in the insert 46e. Also, the strut(s) 50 may extend between arcuate anchors 52, each consisting of a partial circumference of a cylinder shell, as in inserts 46d and 46f. In some embodiments, the struts 50 are not adhered to the elongated tube 12 when mounted thereinto, and only the tubes 48 or arcuate mounts 52 are.

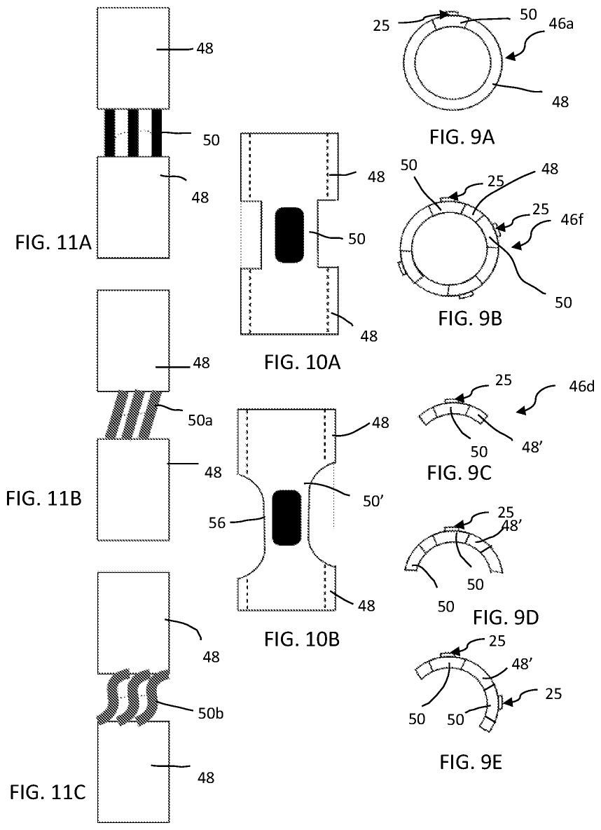

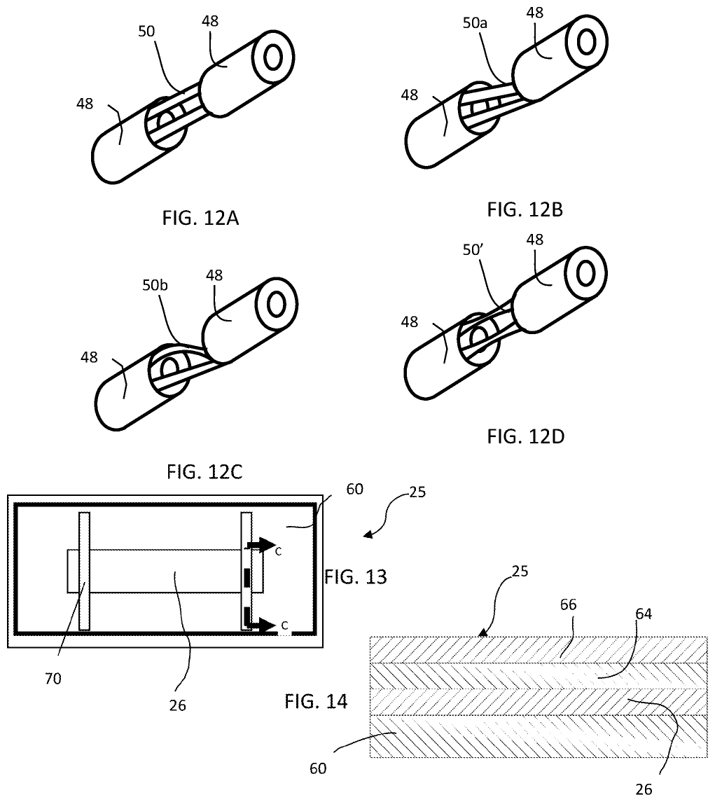

FIGS. 9A to 9E collectively illustrate in transversal cross-sectional views various configurations of anchors 48 and 48′, struts 50 and strain gauges 25. In some embodiments, only one strut 50 and strain gauge 25 combination is provided, as see in FIGS. 9A and 9C. Many strut 50 and strain gauge 25 combinations may be provided circumferentially spaced apart from each other, as seen in FIG. 9B and 9E. Some struts 50 may be devoid of strain gauge 25, as seen in FIG. 9C. The struts 50 may have a constant width longitudinally therealong, as seen in FIG. 10A, or the struts 50′ may have curved edges, typically narrowing centrally, as seen in FIG. 10B, thereby creating a neck 56 between the two anchors 48. Referring respectively to FIGS. 11A to 11C, the struts 50, 50a and 50b may be respectively rectilinear and longitudinal relative to the anchors 48, rectilinear and angled relative to the longitudinal direction, or curved. The struts 50, 50a and 50b are illustrated respectively in FIGS. 12A to 12C, while FIG. 12D illustrates the strut 50′ in embodiments in which two anchors 48 are linked with two struts 50.

In all the above embodiments, relatively thin strain gauges 25 may be manufactured by using a printed conductive trace 26 including a conductive ink that is then dried. The conductive trace 26 can be printed on a relatively thin strips, or on the inserts 46a to 46f or the elongated tube 12 directly. As described in details above, the strips, for example the substrate 54, can be embedded in the main portion, or wall, of the elongated tube 12. In some embodiments, the strain gauge 25 is entirely embedded in the elongated tube 12. This allows manufacturing of an elongated tube in which the inner surface 18 is of substantially constant diameter along the whole deformable segment, or even along the whole elongated tube 12.

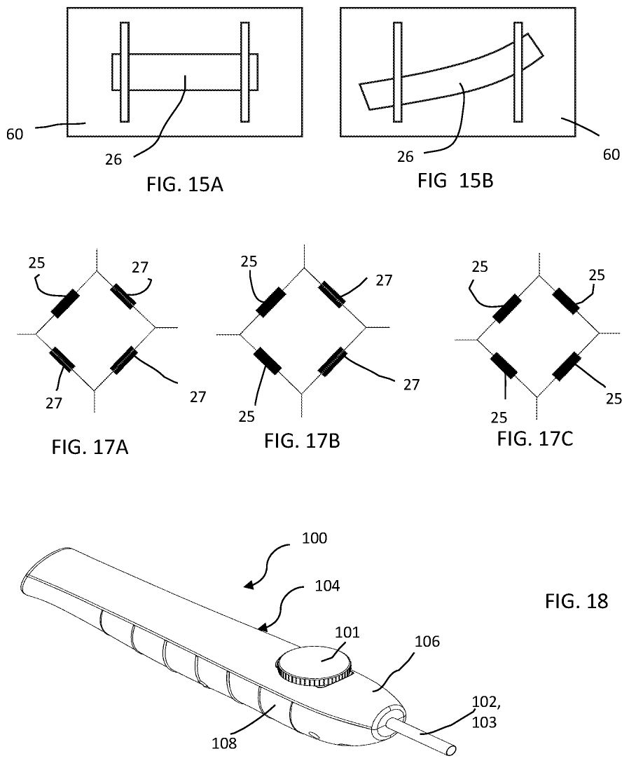

Referring to FIGS. 13 and 14, there is illustrated the layered structure of the strain gauge 25. An electrically isolating substrate layer 60 forms a base of the strain gauge 25. A sensing layer 62 including the conductive trace 26 overlays the substrate layer 60. An interface layer 64 including a pair of conductive contactssignal collectors 70 overlies the conductive trace 26. The signal collectors 70 are conductors that are in electrical connection with the conductive trace 26. Two or more signal collectors 70 extend spaced apart from each other along the conductive trace 26. For example, the interface layer 64 includes silver or any other suitable metal. Finally, an isolating protective layer 66 overlies the interface layer 64. As illustrated respectively in FIGS. 15A and 15B, the conductive trace 26 can be rectangular or curved.

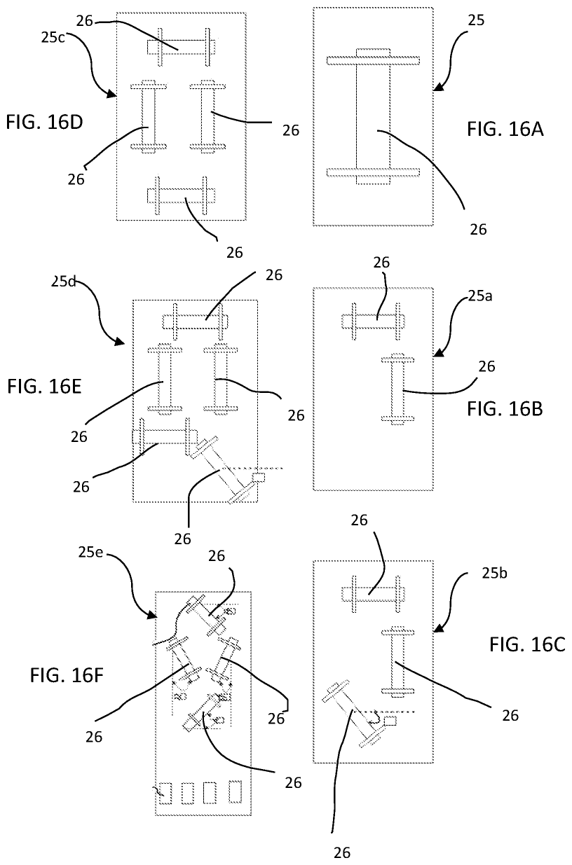

FIGS. 16A to 16F illustrate various configurations of conductive traces 26 usable in various strain gauges 25, and 25a to 25e respectively. Strain gauge 25 includes a single conductive trace 26. Strain gauge 25a includes a pair of conductive traces 26 perpendicular to each other. Strain gauge 25b adds a third conductive trace 26 to the strain gauge 25a that is angled relative to the first two conductive traces 26. Strain gauge 25c includes two pairs of conductive traces 26 perpendicular to each other, with the conductive traces 26 in each pair parallel to each other. Strain gauge 25d adds a fifth conductive trace 26 to the strain gauge 25c that is angled relative to the first four conductive traces 26. Strain gauge 25e includes conductive traces 26 that re mutually non-parallel and non-perpendicular to each other. It should be understood that the number and geometry of the conductive traces 26 and of the strain gauge 25 in general is not limited to what is illustrated in the drawings, and other layouts are usable.

FIGS. 17A to 17C show a variety of circuits that maybe used to measure the changes in the resistance of the sensing layer. Various well known and commercially available circuit architectures maybe used to measure the small changes in the resistance of the conductive traces 26 under loading condition. The Wheatstone bridge, illustrated in FIG. 17A consisting of only one strain gauge 25 and 3 reference resistances 27 can detect the strain at one point, but its output can be impacted by environmental conditions such as temperature variation. To compensate for temperature effect and/or to have higher sensitivity two or four strain gauges 25 can be connected to each other to form half or full bridge configurations as shown in FIGS. 17B and 17C respectively. Some of the strain gauges 25 may share a common ground or a common excitation line to reduce the total number of conductors 32 used in the device 10.

The controller 37 is operative for measuring the resistance, or equivalently the conductance, between pairs of conductors 32 linked to each of the conductive traces 26 using conventional methods. Once these resistances have been measured, they can be processed to infer information regarding the elongated tube 12. Example of information that may be obtained includes a 3D shape of the elongated tube 12 or information about external forces exerted on the elongated tube 12. Such processing can be performed on-board, directly in the controller 37, or in a computer system 200, shown in FIG. 38 and described in further details below.

In some embodiments, the elongated tube 12 is modeled as a beam and the local deformation or strain of a deflected beam is a function of the mechanical properties of the elongated tube 12 and any applied external forces force. Thus, by having the structural specifications of the elongated tube 12 and the magnitude/direction of local deformation at several sensing locations 16, one can determine the 3D shape of the elongated tube and the profile of the forces applied on its outer surface 22. The spatial resolution of the predicted shape and force profiles improves by increasing the number of strain gauges 25, which is practically achievable due to the possibility of manufacturing strain gauges 25 having a relatively footprint.

Generally speaking, using conventional beam modeling theory, one can infer the strain at any location along the elongated tube 12 between and at two longitudinally spaced apart strain gauges 25, which allows determination of a 3D shape of the elongated tube 12, or at least of a section thereof, if enough strain gauges 25 are provided. If external forces are applied between these two strain gauges 25, the magnitude of the resultant force applied on the elongated tube 12 is a function of the two measured strains. The location of the resultant applied force is a function of the difference between the two strains. The functions used to determine the distributed strain along the whole tube, as well as the magnitude and position of external forces can be obtained by a variety of methods, including machine learning, forming a calibration table or look up table, analytical methods, or a combination of these formulations.

In one innovative embodiment, various combinations of shapes and external position and magnitude of forces are applied on the elongated tube 12. For each of these combinations, one can measure the output of all the strain gauges 25 in the elongated tube 12. Then, these couples of (force+shape, strain gauge outputs) can be used to train a neural network. One note that in this embodiment, one can use raw conductance measurements obtained from the strain gauges 25, or calculated strains obtained therefrom. Once the relationship between a standard elongated tube 12 and its shape and external forces applied thereonto has been determined, individual manufactured elongated tubes 12 can have their strain gauges 25 calibrated, and the neural network can then be used to determine the shape and applied external forces in the field, while the elongated tube 12 is in use in surgery.

FIG. 18 illustrates a handle 100 that is usable in replacement of the handle 14 in the device 10. The handle 100 is usable as a controller to control a steerable catheter 102, for example similar in construction to the elongated tube 12. The steerable catheter 102 can also be any steerable catheter 102, such as any conventional steerable catheter including an elongated tube 12 in which tendons 28 (now shown in FIG. 18) are provided. The tendons 28 are either pushed or pulled to deform part of the steerable catheter 102. The handle 100 is, in some embodiments, usable in conjunction with the strain gauges 25 and all the variants thereof described above to assess the shape and forces exerted on the steerable catheter 102. While steerable catheters 102 including only one tendon 28 are usable with the handle 100, the handle 100 is typically used to steer catheters having a pair of tendons 28 acting in opposite directions. In some embodiments, a similar handle may be used to control steerable catheters 102 steerable either along orthogonal planes, or at various inflection points along the steerable catheter.

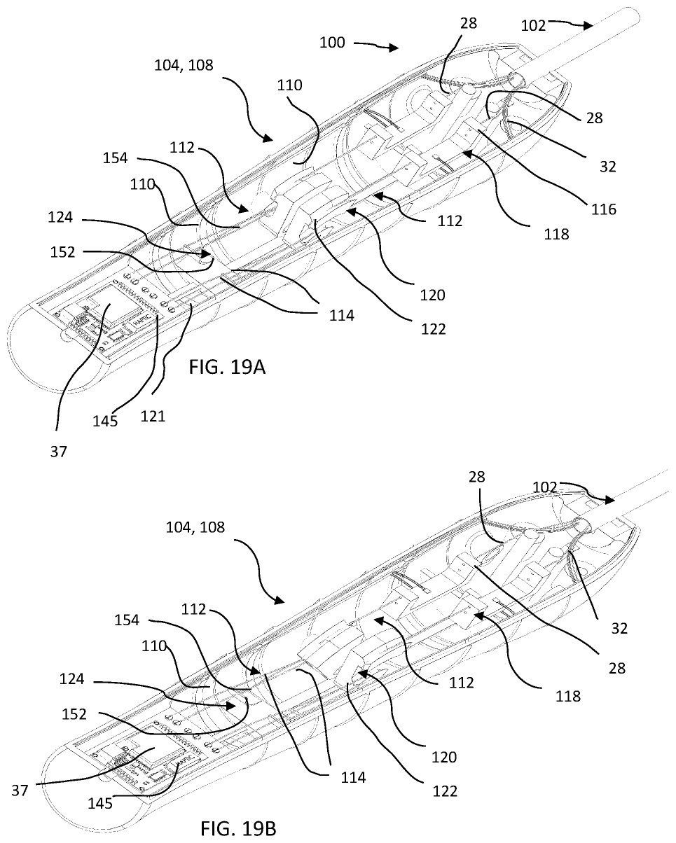

The handle 100 includes a body 104, for example including a pair of shells 106 and 108 joined to each other and defining a cavity 110 (seen in FIGS. 19A and 19B for example) therebetween. The cavity 110 includes the various components that are used to steer the steerable catheter 102. The handle 100 may be powered using a battery or through a conventional power cable (now shown in the drawings).

Referring to FIGS. 19A and 19B, the handle 100 includes a shape memory alloy (SMA) motor 112 mounted to the body 104, and more precisely in the cavity 110. The SMA motor 112 includes a deformable element 114 including a SMA and operable between first and second configurations, seen in FIGS. 19A and 19B respectively, achieved by changing a temperature of the deformable element 114. In some embodiments, the change in configuration is an elongation or shortening of the deformable element 114, by example by a few percent. This change in temperature is typically achieved by through resistive heating of the deformable element 114. Fine control of the deformation between the two extreme configurations of the deformable element 114 can be effected using pulsed heating, such as pulse wave modulation (PWM) or an analog supply. The precision of the deformations provided by the deformable element 114 can be greatly enhanced by using a feedback loop related to the forces exerted on the tendons 28 and/or the deformation of the steerable catheter 102 acquired using strain gauges 25. The SMA motor 112 also has a tendon mount 116 for mounting the tendon 28 thereto. The tendon mount 28 moves relative to the body 104 when the deformable element 114 is moved between the first and second configurations. Thus, with the steerable catheter 102 fixedly mounted to the body 104 and the tendon 28 mounted to the tendon mount 116, moving the deformable element 114 between the first and second configurations moves the tendon 28 relative to the elongated tube 103 to change a configuration of the steerable catheter 102. When more than one tendon 28 are provided, the structures related to their movement are typically duplicated for each tendon 28.

While the general principle of SMA motor 112 described above is relatively simple, the present handle 100 also include the following features enhancing the functionality of the handle 100. Some of these features are omitted in other embodiments. First, a stress sensor 118 is provided in series with the tendon 28 between the tendon 28 and the deformable element 114 to measure a stress in the tendon 28. Also, a transmission 120 is provided between the tendon 28 and the deformable element 114 to change a ratio of displacement between a mobile portion 122 of the deformable element 114 and the tendon 28. Also, a biasing mechanism 124 is provided for biasing the SMA motor 112 in a predetermined direction. These features and the SMA motor 112 are further described in details below.

In steerable catheters 102 of the type including tendon 28 pairs moving the steerable catheter in opposite directions two more SMA motors 112 may be used in opposing configuration to improve the responsiveness of the system. In these embodiments, the handle 100 uses a pair of opposing SMA motors 112 that quickly adjust the tension in tendons 28 in a pull-pull or push-push configuration. Instead of waiting for the temperature of the deformable element to drop before relaxing the tension in the tendon 28, the opposing SMA motor 112 is activated and immediately guides the steerable catheter 102 to the opposite direction.

The SMA motor 112 may be of any known type. For example, as seen in FIGS. 19A and 19B, each SMA motor 112 may include a pair of deformable elements 114 in the form of wires. Each deformable element 114 includes a fixed portion 121, fixedly mounted to the body 104, or to an element thereof that is fixed relative thereto, and the mobile portion 122. The mobile portion 122 moves relative to the body 104 when the deformable element is deformed, so as to pull or push on the tendon 28. The mobile portion 122 can be directly secured to the tendon 28, or, as shown in the drawings, secured to other components of the SMA motor 112, described in greater details below.

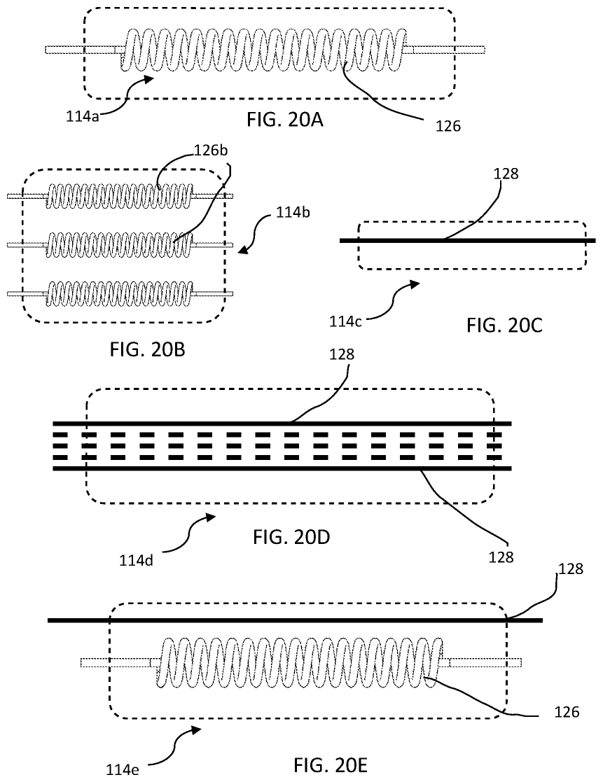

Other configurations for the deformable element 114 are possible, examples of which are shown in FIGS. 20A to 20E. In FIG. 20A, the deformable element 114a includes a spring 126 that has a variable pitch as a function of temperature. As seen in FIG. 20B, an alternative deformable element 114b includes more than one spring 126 in parallel. Alternatively, the deformable element 114c or 114d shown respectively in FIGS. 20C and 20D may include one or more than two deformable wires 128, the wires 128 being in parallel when more than one is present. In yet another embodiment, as shown in FIG. 20E, the deformable element 114e includes both one or more springs 126 and one or more wires 128.

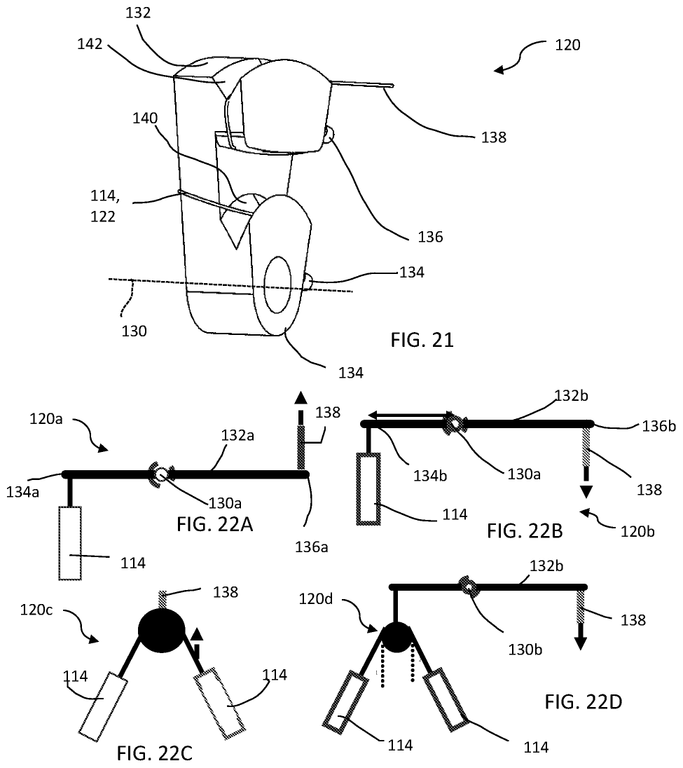

Referring to FIG. 21, the transmission 120 may take the form of a lever component 132 pivotable about a pivot axis 130. The lever component 132 is provided between the deformable element 114 and the tendon 28. The tendon 28 is in some embodiments directly mounted to the lever component 132. However, in the drawings, an intermediate cable 138 is instead secured to the lever component 132. The intermediate cable 138 ultimately transfers forces to the tendon 28, but in the present embodiment, the intermediate cable 138 is secured to the tendon 28 with the stress sensor 118 therebetween.

The lever component 132 defines an input groove 140 receiving the deformable element 114, and more specifically the mobile portion 122, and an input attachment 134 to which the deformable element 114 is fixed so that the deformable element 114 has a portion thereof maintained in the input groove 140. For example, the input attachment 134 includes a post located adjacent the input groove 140, to which the deformable element 114 is mounted. The lever component 132 also defines an output groove 142 receiving the intermediate cable 138 and an output attachment 136 to which the intermediate cable 138 is fixed. For example, the output attachment 136 includes a post located adjacent the output groove 142, to which the intermediate cable 138 is mounted. The intermediate cable 138 is routed so as to remain in the output groove when the lever component 132 is moved by the deformable element 114. The output groove 142 is further away from the pivot axis 130 than the input groove 140 and the input and output grooves 140 and 142 are substantially arcuate and concentric relative to the pivot axis 130. The deformable element 114 and the intermediate cable 138 extend in opposed directions. Therefore, when the deformable element 114 pulls on the lever component 132, the latter pulls on the intermediate cable 138 and increases a magnitude of the movements created by the deformable element 114. In other words, for a given displacement of the mobile portion 122, the intermediate cable 138 will create a larger proportional displacement.

The concentric arrangement of arcuate input and output grooves 140 and 142 allows forces to be exerted perpendicularly to the contact point with the input and output grooves 140 and 142. This maximizes the moment applied by the SMA motor 112 for the lever component 132 a given thereof at all points along the range of motion of the lever component 132. This also provides a linear relationship between the displacement of the tendon 28 and the rotation angle of the lever component 132, which in turn simplifies displacement sensing.

FIGS. 22A to 22D illustrate schematically alternative transmissions 120a to 120d that are usable instead of the transmission 120. As seen in FIG. 22A, the transmission 120a may include a lever 132a having input and output ends 134a and 136a pivotable to a pivot 130a located therebetween. The transmission 120a is coupled to a push deformable element 114 at the input end 134a. The intermediate cable 138 is secured to the output end 136a and extends such that when the deformable element 114 pushes on the input end 134a, the intermediate cable 138 is pulled. As seen in FIG. 22B, the transmission 120b may include a lever 132b having input and output ends 134b and 136b pivotable to a pivot 130b located therebetween. The transmission 120b is coupled to a pull deformable element 114 at the input end 134b. The intermediate cable 138 is secured to the output end 136b and extends such that when the deformable element 114 pulls on the input end 134b, the intermediate cable 138 is pulled. As seen in FIG. 22C, in alternative embodiments the intermediate cable 138 may be coupled to a pair of pull deformable element 114 angled relative to each other so that when both pull deformable elements 114 pull, the intermediate cable 138 is pulled. The arrangement of two pull deformable elements 114 can be used in a configuration similar to FIG. 22B, as seen in FIG. 22D, in which the SMA motor 112 of FIG. 22B is replaced by the arrangement of two angled deformable elements 114, as shown in FIG. 22C.

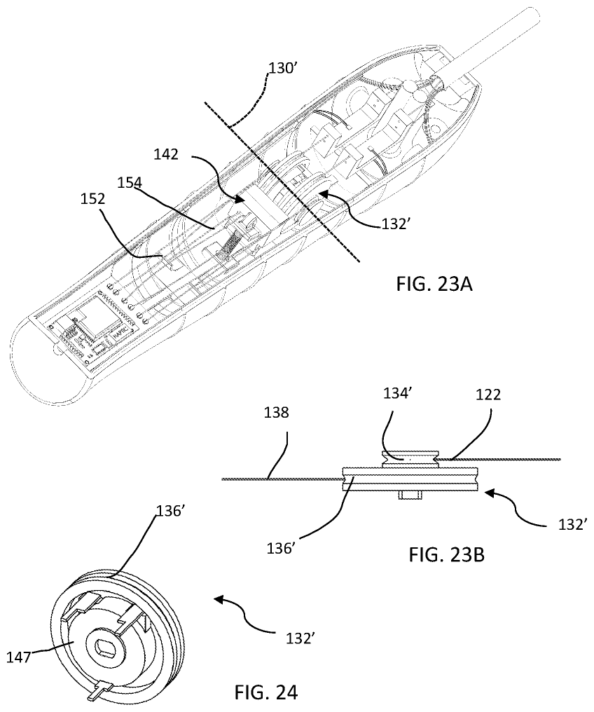

As seen collectively in FIGS. 23A and 23B, in yet other embodiments, the lever component 132 is replaced by a dual grooved pulley 132′ including input and output grooves 134′ and 136′ of different radii around at least part of which the mobile portion 122 and the intermediate cable 138 are wound in opposite direction. The pulley 132′ is rotatable about a rotation axis 130′.

In some embodiments, an angular sensor, such as a potentiometer 147 inside the pulley 132′, as seen in FIG. 24, is provided. The angular sensor provides direct measurement of the displacement effected by the SMA motor 112, and can therefore be used for display purposes, or as part of a feedback loop allowing adjustment of precise tendon 28 displacements. Other motion sensors may be used where the displacement of the tendon 28 is measured by converting the rotational movement of the lever component 132 to a linear one. In one embodiment, Hall sensors maybe used to measure the displacement of a magnet mounted to any movable part of the handle 100, such as the intermediate cable 138 or the SMA motor 112, such that movements of the magnet changes a magnetic field measured by the hall sensor. Other types of movement sensors are also usable, such as inductive or capacitive movement sensors.

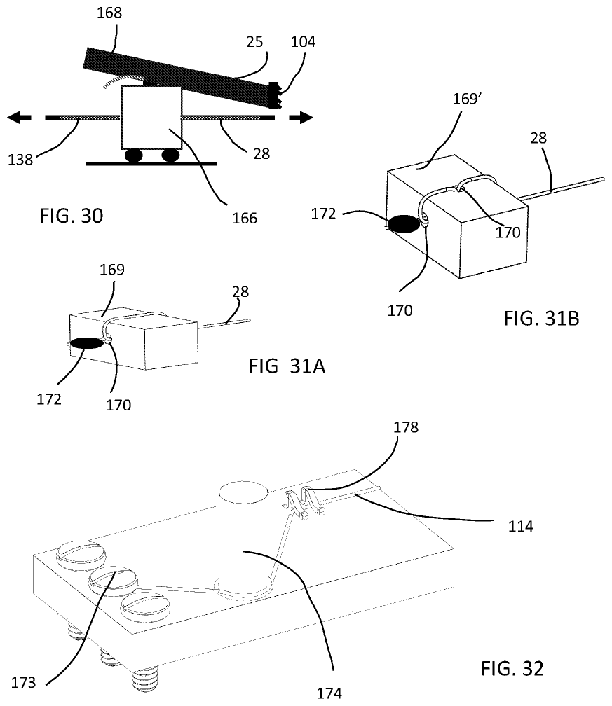

FIG. 30 illustrates yet another manner of determining the displacement of the tendon 28. A guided member 166 is mounted to the tendon 28 and movable jointly therewith. The guided member 166 abuts against a flexible member 168 that is anchored fixedly relative to the body 104 and which extends along the tendon 28. The flexible member 168 is configured and sized so that when the guided member 166 is moved by then tendon 28, when the latter is pulled or pushed, the contact point between the guided member 166 and the flexible member 168 is moved along the guided member 166, which results in deformations of the flexible member 168. Deformations of the flexible member 168 are measured using one or more strain gauge 25. For example, the flexible member 168 takes the form of a flexible beam 168 extending at an acute angle relative to the tendon 28. The contact between the guided and flexible members 166 and 168 can be a simple abutment, or a roller can be provided therebetween to facilitate sliding. In other embodiments, stress is instead measured at the junction between the body 104 and the flexible member 168.

In yet other embodiments, changes in resistance of the deformable element 114 can be used to infer the movements of the tendon 28. Indeed, the martensite and austenite phase or Nickel-Titanium SMAs have different resistivity. Thus, by measuring the resistance of the deformable element, one can get an indication of its deformation, which in turns is indicative of the movements of the tendon 28. In this method, there is no need for another displacement measurement sensor and only an electrical circuit, a data acquisition system, and an algorithm is required to measure the displacement.

Other transmissions allowing to multiply the relatively small displacements provided by the SMA motor 112 are possible. Also, if the lever arm between the intermediate cable 138 and the mobile portion 122 is reversed, displacement will be reduced, but force will be increased by the transmission 120.

Referring to FIG. 29, in some embodiments, a mechanical brake 149 is provided for selectively frictionally engaging the pulley 132′. When enough friction is exerted, the mechanical brake 149 locks the position of the pulley 132′, and therefore of the tendons 28, without requiring further power delivery to the SMA motors 112. The mechanical brake 149 may be activated by a suitable actuator, such as a SMA motor 145 including a deformable element similar to the deformable element 114. A biasing element 143 may bias the mechanical brake 149 in a locked position in which friction locks the pulley 132′. The SMA motor 145 releases the mechanical brake 149 by spacing the mechanical brake 149 from the pulley 132′ by pulling using the SMA motor 145 to achieve an unlocked position. In other embodiment, any other type of actuator is usable for the mechanical brake, such as actuators having high internal friction, as is the case with worm drives, so that when the motor is unpowered, the mechanical brake 149 does not move. In other embodiment, a relay is used.

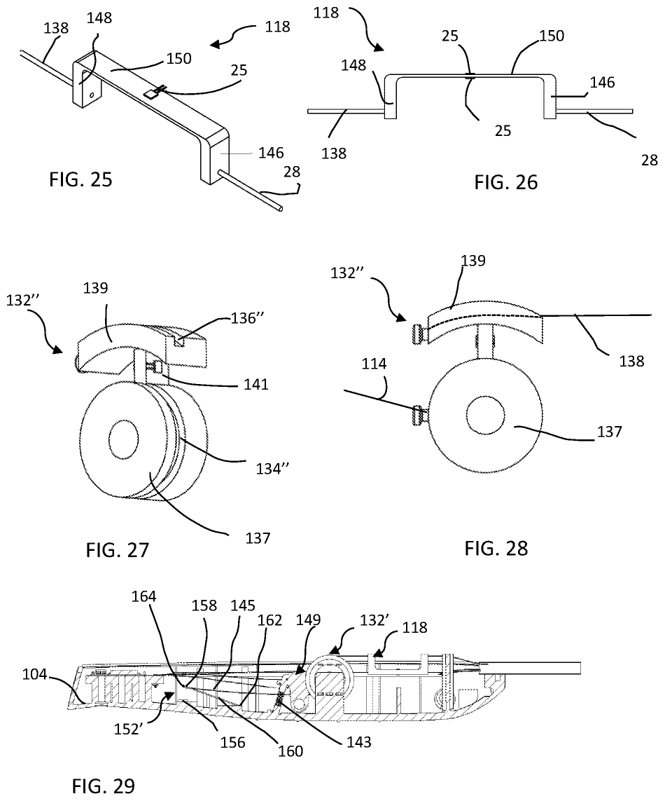

FIGS. 25 and 26 illustrate in greater detail the stress sensor 118. The stress sensor 118 is shaped to deform when a tension is exerted by the SMA motor 112. For example, the stress sensor 118 is substantially U-shaped and includes a pair of parallel end sections 146 and 148 spaced apart from each other by an intermediate section 150 extending generally perpendicular to the end sections 146 and 148. The intermediate cable 138 and tendon 28 are mounted to a respective one of the end sections 146 and 148 spaced apart from the intermediate section 150. Therefore, tension forces in the intermediate cable 138 and tendon 28 tend to bend the intermediate section 150. One or more strain gauges 25 can then be used to measure this bending deformation. The strain gauges 25 can be similar to the strain gauges 25 described above or of any other suitable type. Once the deformation is known, the magnitude of the tension can be deducted from the mechanical properties of the end and intermediate sections 146, 148 and 150. Two strain gauges 25 may be provided on opposite surfaces of the intermediate section 150 to increase sensitivity and to compensate for temperature variations.

The intermediate cable 138 and tendon 28 can be mounted to the end sections 146 and 148 using various methods, including but not limited to making a knot, glue and wrapping around screws, among others. The deformable U-shaped substrate can be made of various materials including but not limited to metals, plastics and rubbers, among others. Other types of stress sensors are also usable, including, but not limited to, rectilinear stress sensors including an elongated beam of constant or variable width therealong, V-shaped stress sensors, stress sensors that are guided along a predetermined path when the tendon 28 moves.

FIGS. 27 and 28 collectively illustrate an alternative lever component 132″ that integrates the transmission functionality and the force sensing functionality. The lever component 132″, instead of being a relatively bulky and solid structure includes a deformable section between the input and output grooves 134″ and 136″. For example, the input groove 134″ is formed on a generally cylindrical component 137, from which a deformable radial member 141 extend, which is terminated by an arcuate component 139 defining the output groove 138″. When forces are exerted by the deformable element 114, displacement of the intermediate cable 138 will be provided, as with the lever 132, but the radial member 141 will deform. Such deformations can be detected using strain gauges 25 and are indicative of the force exerted by the deformable element.

In some embodiments, referring to FIG. 19A for example, a biasing mechanism 124 is provided for biasing the deformable element 114 in a predetermined direction, for example by pre-tensing the deformable element 114 so that a pulling force is exerted on the tendon 28 by the SMA motor 112 even without heating the deformable element 114. The biasing mechanism 124 may for example include leaf springs 152 mounted to the body 104 and biasing cables 154 extending between the leaf springs 152 and the lever component 132 or 132″ or the pulley 132′. The length of the biasing cables 154 is such that the leaf springs 152 are biased when the deformable elements 114 are in a predetermined configuration, for example, and non-limitingly, in a neutral configuration corresponding to a straight catheter 102, among other possibilities. Other biasing components, such as helical springs are usable.

Referring to FIG. 29, in other embodiments, a magnetic spring 152′ is used to provide the bias force. In this configuration, a stationary magnetic component 156, such as a magnet or ferritic block, is mounted to the body 104 and attracts a mobile magnetic component 158, also a magnet or ferritic block, attached to an elongated flexible member 160. The flexible member 160 is anchored to the body 104 at a fixed end 162, between the stationary magnetic component 156 and the lever component 132 (not shown in FIG. 29) or pulley 132′. The mobile magnetic component 156 is provided at a free end 164 of the flexible member 160, adjacent the stationary magnetic component 156. The biasing cable 154 is anchored also at the free end 164. Therefore, the two magnetic components 156 and 158 will attract each other, which will increase a tension in the tendon 28 as a function of position and shape of the deformable element 114 without requiring large forces to be exerted by the latter. This configuration may therefore reduce the overload of the biasing mechanism 124 at higher loads while providing enough bias force at lower temperature. Other mechanical structures having equivalent function can replace the flexible member 160.

In some embodiments, a haptic engine 145, seen in FIG. 19A for example, is provided in the handle 100 to provide tactile feedback to the intended user, for example by being activated when predetermined forces are exerted on the catheter 102, measured as defined hereinabove for the elongated tube 12.

The various elongated flexible components of the device 10 and handle 100 can be secured to other components in any suitable manner, for example using knots, crimping, adhesives, screws, nut and bolt combinations, among others. FIGS. 31A and 31B illustrate two relatively solid manners of attaching such members, here illustrated with attachment of a tendon 28 in a block 169. Referring to FIG. 31A, a through aperture 170 extends through the block 169. The tendon is looped through the aperture 170 and an anchor, such as a clamp 172 or a spot of glue larger than the aperture 170 is mounted to the tendon 28, so that the tendon 28 can no longer be pulled through the aperture 170. As seen in FIG. 31B, additional apertures 170 can be provided to form additional loops. The loops help in dissipating forces exerted by the tendon 28 on the clamp 172.

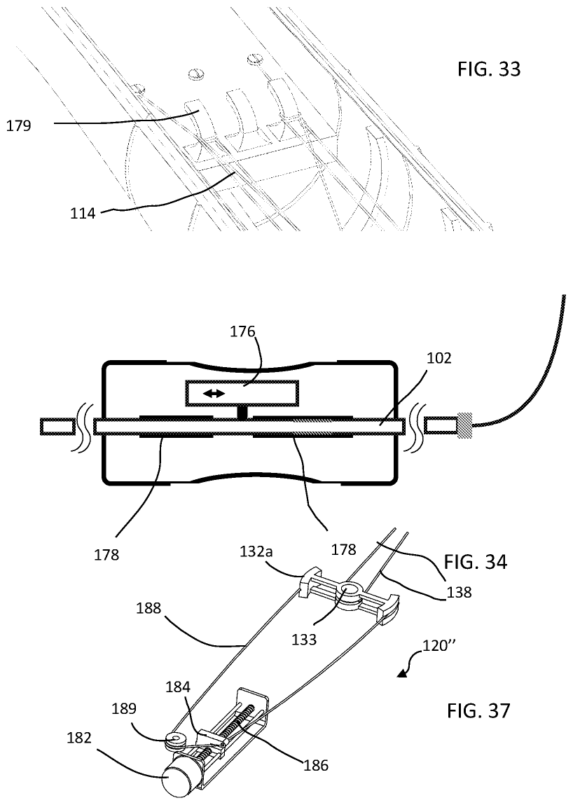

FIG. 32 illustrates another manner of dissipating pulling forces so that an anchoring point required less force than for a direct anchor. An elongated wire, for example the deformable component 114 of the SMA motor 112 may be rolled around a post 174 and then anchored using a screw 173. A guide adjacent the post 174 may receive the deformable component so as to have a portion thereof that maintains a fixed position. In some embodiments, as seen in FIG. 33, a single deformable element 114 can be rolled around posts 179 and suitable attachments on the transmission 120 (not shown in FIG. 33), so that many sections thereof extend in parallel between the post 179 and the transmission 120. Therefore, a relatively large force can be exerted using a single component, similar to what would occur if many deformable elements were used in parallel.

In some embodiments, as shown in FIG. 34, a piezo motor 176 frictionally engaging the catheter 102 is used to move the latter longitudinally along guides so that the catheter 102 can be advanced precisely.

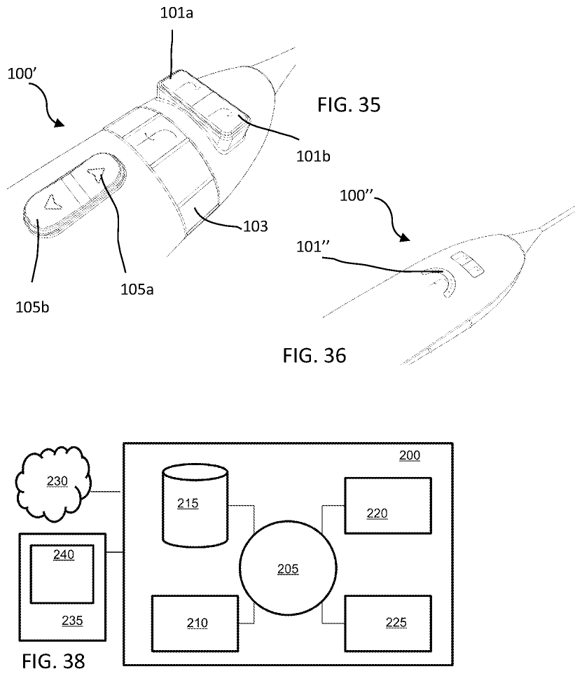

The handle 100 includes a rotary knob 101, seen in FIG. 19A for example, to control steering of the steerable catheter 102. Rotating the knob 101 steers the steerable catheter 102 in the direction of rotation. Other interfaces are also possible, non-limiting examples of which are described below. FIG. 35 illustrates an alternative handle 100′ including a pair of push-buttons 101a and 101b side-by-side. Pressing one of the push buttons 101a or 101b steers the catheter in the corresponding direction. Optionally, a circumferential knob 103 is provided to rotate the steerable catheter 102 about its longitudinal axis. Furthermore, longitudinally spaced apart push buttons 105a and 105b are usable to control longitudinal movements of the steerable catheter 102 in corresponding directions when such movements are enabled. FIG. 36 illustrates an alternative handle 100″ including an arcuate tactile sensor 101″. Moving a finger along the tactile sensor 101″ steers the steerable catheter 102 in the corresponding direction. These features of user interface can be mixed to each other or individually provided, depending on the embodiment.

As shown in FIG. 37, other types of motors can be used instead of the deformable element 114. For example, FIG. 37 illustrates an embodiment in which the deformable element 114 is replaced by an electric motor 182 operating an endless screw 186 to which a carriage 184 is mounted so at to move along the endless screw 186 when the latter is rotated. The transmission 120″ includes a lever 132a pivotable about central pivot. Two intermediate cables 138 are mounted to the lever 132a on opposed sides of the pivot 133 to control two tendons 28 (not shown in FIG. 37) that operate in tandem to steer a catheter. In alternative embodiments, any of the other transmissions described herein can be used with a similar actuator. An actuating cable 188 has both of its end secured to the lever 132a on opposed sides of the central pivot 133. The actuating lever is routed through a pulley and has a portion thereof secured to the carriage 184. Thus, when the carriage 184 moves along the endless screw 186, the actuating cable rotates about the pulley, which in turn rotates the lever 132a. Due to the relatively large movement range achievable using the endless screw 186, the mechanical advantage provided with the lever 132a can be reversed compared to the above-described transmissions, so that force is multiplied and displacement is demultiplied at the output of the lever 132a, namely the intermediate cables 138. Other components of the system remain unchanged and are not further described.

In some embodiments, the device 10 and all the variants described in the present document may be controlled locally or remotely over a network using a dedicated controller or a computer system 200. An example of such a computer system 200 is shown in FIG. 38. The computer system 200 that is programmed or otherwise configured to operate or interface with the device 10. The computer system 200 may be used to steer, rotate or translate the catheter 102 and obtain measurements from the various strain gauges 25. In some embodiments, the computer system 200 is embedded in the controller 37 present in the handle.

The computer system 200 includes a central processing unit (CPU, also “processor” and “computer processor” herein) 205, which can be a single core or multi core processor, or a plurality of processors for parallel processing. The computer system 200 also includes memory or memory location 210 (e.g., random-access memory, read-only memory, flash memory), electronic storage unit 215 (e.g., hard disk), communication interface 220 (e.g., network adapter) for communicating with one or more other systems, and peripheral devices 225, such as cache, other memory, data storage and/or electronic display adapters. The memory 210, storage unit 215, interface 220 and peripheral devices 225 are in communication with the CPU 205 through a communication bus (solid lines), such as a motherboard. The storage unit 215 can be a data storage unit (or data repository) for storing data. The computer system 200 can be operatively coupled to a computer network (“network”) 230 with the aid of the communication interface 220. The network 230 can be the Internet, an internet and/or extranet, or an intranet and/or extranet that is in communication with the Internet. The network 230 in some cases is a telecommunication and/or data network. The network 230 can include one or more computer servers, which can enable distributed computing, such as cloud computing. The network 230, in some cases with the aid of the computer system 200, can implement a peer-to-peer network, which may enable devices coupled to the computer system 200 to behave as a client or a server.

The CPU 205 can execute a sequence of machine-readable instructions, which can be embodied in a program or software. The instructions may be stored in a memory location, such as the memory 210. The instructions can be directed to the CPU 205, which can subsequently program or otherwise configure the CPU 205 to implement methods of the present disclosure. Examples of operations performed by the CPU 205 can include fetch, decode, execute, and writeback.

The CPU 205 can be part of a circuit, such as an integrated circuit. One or more other components of the system 200 can be included in the circuit. In some cases, the circuit is an application specific integrated circuit (ASIC).

The storage unit 215 can store files, such as drivers, libraries and saved programs. The storage unit 215 can store user data, e.g., user preferences and user programs. The computer system 200 in some cases can include one or more additional data storage units that are external to the computer system 200, such as located on a remote server that is in communication with the computer system 200 through an intranet or the Internet.

In some embodiments, the computer system 200 can communicate with one or more remote computer systems through the network 230. In some embodiments, the peripheral devices 225 include a dedicated interface 245 operable to communicate with the controller 37, which can receive commands from the computer system 200 and use electronic component to actuate the SMA motor 112. The controller 37 can also receive signals from the strain gauges 25 and send corresponding data to the computer system 200 for processing. This dedicated interface may be provided in a separate device connected to the remainder of the computer system 200 through an interface, for example and non-limitingly a USB bus. The dedicated interface 245 may also be integrated on a card inserted in a suitable slot of a motherboard, such as a PCI slot, non-limitingly. Examples of remote computer systems include personal computers (e.g., portable PC), slate or tablet PC's (e.g., Apple® iPad, Samsung Galaxy Tab), telephones, Smart phones (e.g., Apple® iphone, Android-enabled device, Blackberry®), or personal digital assistants. The user can, in some embodiments, access the computer system 200 via the network 230.

Control of the steerable catheter 102 and/or acquisition of data from strain gauges is effected through executable code stored on an electronic storage location of the computer system 200, such as, for example, on the memory 210 or electronic storage unit 215. The machine executable or machine readable code can be provided in the form of software. During use, the code can be executed by the processor 205. In some cases, the code can be retrieved from the storage unit 215 and stored on the memory 210 for ready access by the processor 205. In some situations, the electronic storage unit 215 can be precluded, and machine-executable instructions are stored on memory 210.

The code can be pre-compiled and configured for use with a machine having a processer adapted to execute the code, or can be compiled during runtime. The code can be supplied in a programming language that can be selected to enable the code to execute in a pre-compiled or as-compiled fashion. The code may provide a user interface allowing commands to be entered, and a control module controlling the dedicated interface, for example in the form of a device driver.

The computer system 200 can include or be in communication with an electronic display 235 that comprises a user interface (UI) 240 for providing, for example, controls corresponding to the various movements that the steerable catheter 102, which may also, either in the alternative or in combination, be controlled using the various push buttons, knobs and tactile sensors described above.

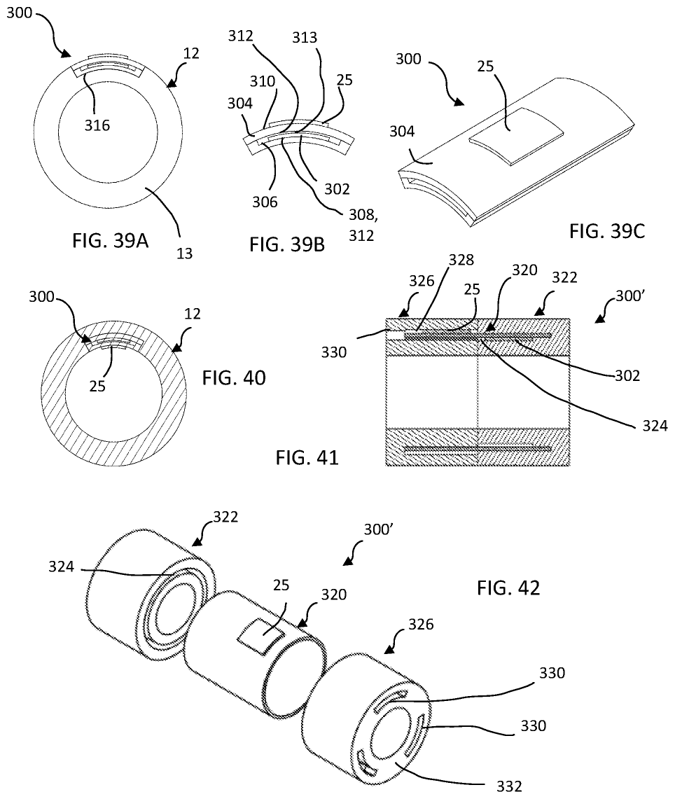

It has been found that, surprisingly, strain gauges 25 including a printed conductive trace 26 as described above are very sensitive to pressure and are usable as a pressure sensor intrinsically, as opposed to being used to measure deformation of a flexible element in response to pressure as is conventional. Thus, referring collectively to FIGS. 39A, 39B and 39C, the elongated tube 12 can be equipped with a pressure sensor 300 including the strain gauge 25 and a temperature compensator 302 enclosed in a casing 304.

More specifically, the pressure sensor 300 includes the casing 304, which defines an enclosed cavity 306 delimited by an internal surface 308 and an outside surface 310 opposed to the internal surface 308. The strain gauge 25 used to sense pressure is secured to the outside surface 310. The temperature compensator 302 is provided in the cavity 306 so as to be immune from pressure variations. For example, the temperature compensator is in the form of a strain gauge 25 secured in the cavity 306 to an attachment section 312 part of the internal surface 308 of the cavity, so that the second strain gauge 25 has an exposed surface 313 spaced apart from a spaced section 314 of the internal surface 308 facing the attachment section 312. In other words, there is a gap between the exposed surface of the second strain gauge 25 and the internal surface 308. In other embodiments, the cavity 306 is filled with a filler, such as an epoxy or other suitable material.

Pressure exerted on the first strain gauge 25 produces changes in conductance of its conductive trace 26. In some embodiments, the two strain gauges 25 are part of a half Wheatstone bridge. The strain gauges 25 are secured to the casing 304 in any suitable manner, for example using an adhesive. It should be understood that if suitable circuitry is provided, the temperature compensator 302 could also be used as a thermometer to provide values for the environmental temperature. Also, other suitable means for compensating the changes in electrical properties of the strain gauge 25 in response to changes in temperature are usable.

In some embodiments, the temperature compensator 302 is a strain gauge 25 of the construction described above encased in a rigid casing 304. The temperature compensator 302 is provided in a cavity 306 formed in the casing 304. Therefore, the only variable that will affect the temperature compensator is the temperature, as the temperature compensator is immune from pressure variations. If a pressure insensitive temperature compensator 302 is used, it is not necessarily enclosed in a cavity can be exposed.

The casing 304 may be received in a recess 316 formed in the wall 13 of the elongated tube 12. The strain gauge 25 may be covered if desired with a protective film or membrane, as the strain gauge 25 may exposed to biological fluids, such as blood, to perform its measurements. Typically, the casing 304 is shaped so that once inserted in the recess 316, the casing 304 merges smoothly with adjacent portions of the elongated tube 12. In FIG. 39A, the pressure sensor 300 has its sensing strain gauge 25 provided facing outside the elongated tube 12. In other embodiments, as seen in FIG. 40, the pressure sensor 300 has its sensing strain gauge 25 facing inwardly, in the lumen of the elongated tube 12.

In a specific embodiment of the invention, the casing 304 includes a pair or arcuate shells separated from each other by a pair of circumferentially opposed side walls. The casing 304 may be closed at its longitudinal ends. In other embodiments, the casing 304 is open at its longitudinal ends, and the cavity 306 is closed at these ends by the bulk of the wall 13 of the elongated tube 12. The casing 304 is typically relatively rigid, so that pressure sensing can be distinguished from bending deformation of the casing 304. In alternative embodiments, the casing 304 may be parallelliped-shaped or have any other suitable shape.

FIGS. 41 and 42 illustrate yet another pressure sensor 300′ that is stand-alone, that is not manufactured as an integral part of the elongated tube 12. The pressure sensor 300′ can then be integrated into a catheter by being inserted in its lumen, or used in any other suitable manner. The pressure sensor 300′ includes the strain gauge 25 and the temperature compensator 302, which are mounted respectively outside and inside a cylindrical shell 320, at longitudinally spaced apart locations.

A first tubular end piece 322 defines an annular groove 324 extending longitudinally thereinto from an end surface thereof, which received a portion, for example, half, of the cylindrical shell 320. The annular groove 324 is configured and sized such that the temperature compensator 302 is completely received thereinto and is closed at its bottom end. A second tubular end piece 326 also defines an annular groove 328 extending longitudinally thereinto, which receives a portion, for example, half, of the cylindrical shell 320. The annular groove 328 is configured and sized such that the strain gauge 25 is completely received thereinto. The first and second end pieces 322 and 326 have annular grooves 324 and 328 such that the second end piece 326 closes the open end of the annular groove 324 of the first end piece 322 when the first and second end pieces 322 and 326 are mounted to the cylindrical shell 320. When assembled, the cylindrical shell 320 is enclosed in the two end pieces 322 and 326.

One or more sensing apertures 330 extend into the second end piece 326 and reach its annular groove 328, so that fluids present in the environment of the pressure sensor 300′ can transmit their pressure to the strain gauge 25, which has an exposed surface in the annular groove 328. For example, these sensing apertures 300′ extend longitudinally in prolongation of the annular groove 326, so that sensing occurs through an end face 332 of the second end piece 328.

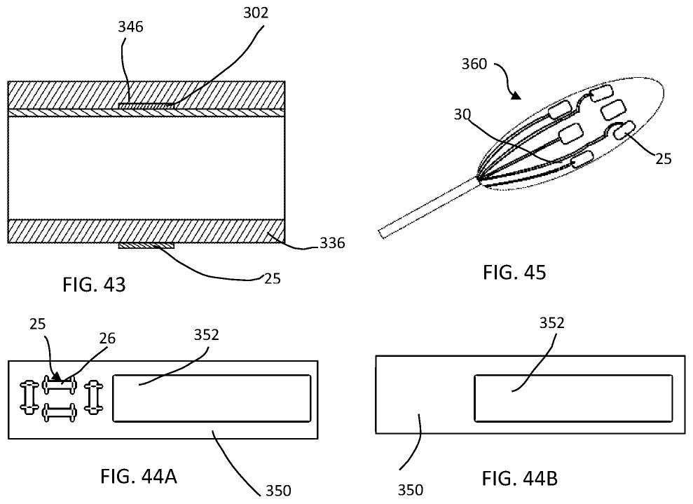

As seen in FIG. 43, in other embodiments, the temperature compensator 302 may be provided in a distinct cavity 334 formed in a body 336 to which the strain gauge 25 is attached, exposed to the environment.

PVDF piezo sensors are widely used in various applications for their high sensitivity and flexibility. However, because of their capacitive nature, these sensors are high pass filters which attenuate and distort signals below their cutoff frequency. The capacitance of the sensor and the attached resistive loads are usually chosen to reach the desired cutoff frequency. However, the design space is limited for various reasons including the limitation on the available footprint and the signal to noise ratio. A wideband sensor may be used using the technologies disclosed in this document. Indeed, the printing/deposition manufacturing process utilized in manufacturing PVDF piezo sensors and the strain gauge 25 disclosed in this document can be used to achieve a wideband sensor. FIGS. 44A and 44B illustrate an example of such embodiments comprising a plate-shaped shared PVDF piezo substrate 350, piezo conductive planes 352, for example made of silver, provided on opposed sides of the substrate 350, and one or more strain gauge(s) 25, four of which are shown in FIG. 44A. The stain gauges 25 includes conductive traces 26 that are printed on the substrate 350. The silver layer may be deposited, based on the requirements, at the same stage as the conductive plane of the piezo sensor. The signals from the PVDF sensor and the strain gauges 25 can be extracted with various methods.

The proposed sensing technology is usable in other applications also. Strain gauges 25 can be mounted on an angioplasty balloon 360, as seen in FIG. 45, to assess its shape.

Also, it has been shown that the quality of some minimally invasive procedures, such as ablation, improves by maintaining a constant contact force between the tip of the device and the heart tissue. Using the proposed instrumented device 10 as described above for force sensing may allow one to determine the force exerted on the tip of the device. Such force measurements may be used in a feedback loop to maintain a predetermined pressure on tissue at the tip of the elongated tube 12, which may be equipped for example with electrosurgery electrodes. Such contact can be maintained by using the tendons 28 to maintain the proper force, without the need for a surgeon to adjust for tissue movement.

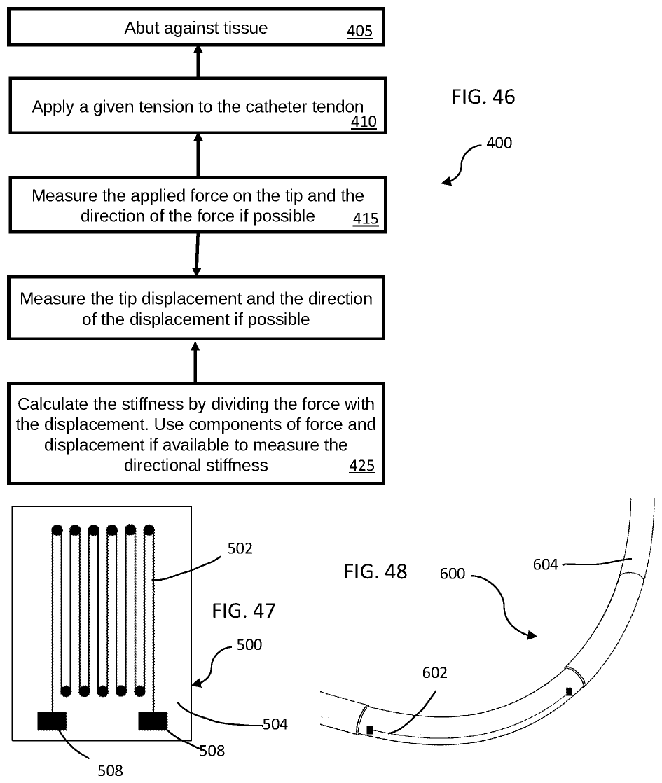

FIG. 46. illustrates an algorithm 400 to measure the stiffness of a tissue using the instrumented device 10. First, at step 405, one abuts a tip of the elongated tube 12 against the tissue to assess. Then, at step 410, one applies a given tension to one of the tendon 28, at step 415, one measures the applied force on the tip and the direction of the force in some embodiments. At step 420, one measures also the corresponding tip displacement and the direction of the displacement in some embodiments. One can then calculate the stiffness by dividing the force with the displacement, at step 425.

In other embodiments, a SMA wire made of a shape memory Nickel-Titanium alloy may be used as a strain gauge, as seen in FIG. 47. Indeed, in addition to their shape memory properties, SMA materials have a well-known superelastic behavior where the material can undergo 5-7% of strain without being plastically deformed. During this phase transformation induced process the resistivity of the material also changes. For example the resistivity of NiTi in austenitic and martensitic phases are, respectively, 82×10⁻⁶Ω·cm and 76×10⁻⁶Ω·cm. This change in resistivity helps increase the sensitivity of the SMA based strain gauges from around 2 to a range between 2.5 to 4.5. In this embodiment the shape memory alloy is trained to be in the super elastic region in operational temperature of the strain gauge. The initial condition of the gauge traces or wire is designed to be in the phase transition range where the stress-strain curve plateaus. The resistance of the strain gauge changes upon deforming the traces because of i) geometrical change similar to the conventional gauges and ii) change in resistivity because of the phase transformation. The resulting strain gauge 500 may include a SMA wire 502 that is routed in the conventional zigzagging pattern of parallel lines on a substrate 504, for example using posts 506, conductive pads 508 are electrically connected to the SMA wire 502 to measure its resistance, which will depend on the deformation of the SMA wire 402. In alternative embodiments, SMA wire may be laminated or secured to a flexible substrate. The substrate may be attached to the measurement object by glue or other means used for installing strain gauges. In yet other embodiments, the SMA wire 502 is traced or deposited on the substrate.

FIG. 48 shows an example of an instrumented catheter 600 that uses a SMA based sensing element 602 to detect the strain in the deformable section thereof. The sensing element 602 can be made from a single thin wire attached on the two ends to the wall 604 of the catheter 600.

Although the present invention has been described hereinabove by way of exemplary embodiments thereof, it will be readily appreciated that many modifications are possible in the exemplary embodiments without materially departing from the novel teachings and advantages of this invention. Accordingly, the scope of the claims should not be limited by the exemplary embodiments, but should be given the broadest interpretation consistent with the description as a whole. The present invention as described can be modified without departing from the spirit and nature of the subject invention as defined in the appended claims.

1. An instrumented minimally invasive surgical device, comprising:
an elongated tube defining an inner surface delimiting a lumen and an opposed outer surface, the elongated tube including a deformable segment; and
a strain gauge, the strain gauge including a conductive trace provided in the elongated tube between the inner and outer surfaces in the deformable segment, wherein a conductance of the conductive trace depends on a deformation of the deformable segment.

2. The instrumented minimally invasive surgical device as defined in claim 1, wherein the conductive trace is a printed conductive trace including a dried or cured conductive ink.

3. The instrumented minimally invasive surgical device as defined in claim 2, wherein the elongated tube includes a wall, and wherein the strain gauge includes a strip embedded in the wall, the printed conductive trace being printed on the strip.

4. The instrumented minimally invasive surgical device as defined in claim 1, wherein the strain gauge is entirely embedded in the elongated tube.

5. The instrumented minimally invasive surgical device as defined in claim 1, wherein the inner surface is of substantially constant diameter along the whole deformable segment.

6. The instrumented minimally invasive surgical device as defined in claim 2, wherein the elongated tube includes a wall extending along the whole deformable segment and wherein a strip is bonded to the wall, the printed conductive trace being printed in the strip, the strip defining part of the inner surface.

7. (canceled)

8. The instrumented minimally invasive surgical device as defined in claim 1, further comprising a pair of conductors electrically connected to the conductive trace spaced apart from each other, the conductors extending along the elongated tube to a free end thereof.

9. The instrumented minimally invasive surgical device as defined in claim 8, further comprising a controller operatively coupled to the conductors for measuring a resistance between the two conductors, wherein the resistance is indicative of a deformation of the elongated tube.

10. (canceled)

11. The instrumented minimally invasive surgical device as defined in claim 10, wherein the controller is operative for outputting a 3D shape of the deformable segment when fed with the respective resistances.

12. The instrumented minimally invasive surgical device as defined in claim 10, wherein the controller is operative for outputting at least one of a magnitude, orientation and location of an external contact force exerted on the deformable segment when fed with the respective resistances.

13. (canceled)

14. The instrumented minimally invasive surgical device as defined in claim 8, wherein the elongated tube is a steerable tube provided with one or more actuating tendons extending through one or more tendon lumen provided in the elongated tube.

15. The instrumented minimally invasive surgical device as defined in claim 14, wherein the conductors extend through the one or more tendon lumen.

16. The instrumented minimally invasive surgical device as defined in claim 1, wherein the strain gauge has a layered structure including:
an isolating substrate layer;
a sensing layer including the conductive trace and overlying the substrate layer;
an interface layer including a pair of conductive contacts overlying the conductive trace in electrical connection therewith opposed to the substrate layer; and
an isolating protective layer overlying the interface layer.

17. (canceled)

18. (canceled)

19. (canceled)

20. The instrumented minimally invasive surgical device as defined in claim 1, further comprising a temperature compensation trace.

21. A controller for a steerable catheter, the steerable catheter having an elongated tube and a tendon operatively coupled to the elongated tube for steering the steerable catheter, the controller comprising
a body;
a shape memory alloy (SMA) motor mounted to the body, the SMA motor including a deformable element including a SMA and operable between first and second configurations achieved by changing a temperature of the deformable element, the shape memory alloy motor having a tendon mount for mounting the tendon thereto, the tendon mount moving relative to the body when the deformable element is moved between the first and second configurations;
wherein, with the elongated tube fixedly mounted to the body and the tendon mounted to the tendon mount, moving the deformable element between the first and second configurations moves the tendon relative to the elongated tube to change a configuration of the catheter.

22. The controller as defined in claim 21, further comprising a stress sensor in series with the tendon between the tendon and the deformable element to measure a stress in the tendon.

23. The controller as defined in claim 22, further comprising a transmission between the tendon and the deformable element to change a ratio of displacement between a mobile portion of the deformable element and the tendon while keeping the same moment arm at any point of actuation.

24. (canceled)

25. The controller as defined in claim 23, wherein the transmission includes a pulley, the controller further comprising an angular sensor for sensing rotations of the pulley relative to the body.

26. A pressure sensor, comprising:
a casing defining an enclosed cavity defining an outside surface;
a first strain gauge secured to the outside surface, the first strain gauge including a first conductive trace made of a conductive ink deposited on a first substrate;
a second strain gauge similar to the first strain gauge and provided in the cavity so as to be shielded from external pressure variations;
wherein pressure exerted on the first strain gauge produce changes in conductance of the first conductive trace.

27. The pressure sensor as defined in claim 26, wherein the first and second strain gauges are part of a half-Wheatstone bridge.

28. (canceled)

29. (canceled)

30. (canceled)
